(19) United States
(12) Patent Application Publication
(10) Pub. No.: US 2024/0238572 A1
(43) Pub. Date: Jul. 18, 2024

(54) Vascular Access Device with Over-the-Catheter Introducer Needle and Near Patient Access Port

(71) Applicant: Becton, Dickinson and Company, Franklin Lakes, NJ (US)

(72) Inventor: Jonathan Karl Burkholz, Salt Lake City, UT (US)

(21) Appl. No.: 18/154,152

(22) Filed: Jan. 13, 2023

(51) Int. Cl. A61M 39/02; A61M 25/06

(52) U.S. Cl. CPC A61M 39/02; A61M 25/0606; A61M 2039/0202

(57) ABSTRACT

Provided herein is a vascular access device that includes a catheter insertion apparatus and a needleless access connector unit. The catheter insertion apparatus includes a housing defining a passage, a needle slideably mounted within the passage and defining a needle lumen, and a catheter assembly having a frame and a catheter coupled to the frame, with the frame sliding within the housing to advance the catheter within the needle lumen. The catheter insertion apparatus is operable in an initial configuration where the needle extends distally from the housing and the catheter is proximally retracted within the needle lumen, and in an actuated configuration where the needle is retracted back into the passage of the housing and the catheter extends distally from the housing. The needleless access connector unit is positioned at a proximal end of the frame of the catheter assembly and provides an access port to the catheter.

### BACKGROUND OF THE INVENTION

### Field of the Invention

Provided herein are devices and systems for use in vascular access, and, in particular, devices for catheter insertion configured to also provide vascular access for instrument delivery and blood draw device use.

### Description of Related Art

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In utilizing a VAD with a patient, a needle is used to access a vein or artery in the body in order to introduce the catheter into a blood vessel. Common catheter insertion techniques include “over-the-needle” and “over-the-catheter” techniques. In an “over-the-needle” technique, a catheter insertion apparatus is employed that operates to insert a needle through the skin and into the blood vessel until the needle tip is properly located in the vessel. After the needle is in proper position, the catheter insertion apparatus operates to advance a catheter over the needle and into the vessel, i.e., the catheter is “over-the-needle,” and the needle is then subsequently withdrawn from the body, leaving the catheter implanted with the distal end of the catheter located in the vessel. In an “over-the-catheter” technique, a catheter insertion apparatus is employed that operates to insert a needle through the skin and into the blood vessel until the needle tip is properly located in the vessel. After the needle is in proper position, the catheter insertion apparatus operates to advance a catheter through the needle and into the vessel, i.e., the needle is “over-the-catheter,” and the needle is then subsequently withdrawn from the body, leaving the catheter implanted with the distal end of the catheter located in the vessel.

Each of the catheter insertion techniques described above has limitations associated therewith regarding the withdrawal of the needle and subsequent removal of the catheter insertion apparatus from the insertion site. With an over-the-needle type catheter insertion apparatus, the proximal portion of the catheter insertion apparatus is typically completely disconnected from a catheter hub that remains secured to the patient at the insertion site. With an over-the-catheter type catheter insertion apparatus, the needle cannot be easily removed from the catheter and disposed of because of the interference of the catheter hub at a proximal end thereof, thereby typically requiring use of a splittable needle. In either case, the structure of existing catheter insertion apparatuses does not allow for an entirety of the catheter insertion apparatus to remain intact at the insertion site and allow for the simple connection of other associated devices (e.g., introducers) thereto by which a guidewire, sensor, wires, fiber, obturator, or other instrument may be introduced through the catheter insertion apparatus (and through the catheter), into the vasculature of the patient.

Accordingly, a need exists in the art for a VAD that provides for catheter insertion and for vascular access for instrument delivery and blood draw. The VAD would desirably include an over-the-catheter introducer needle and a near-patient access port that is integrated therewith or coupleable thereto, through which such instrument delivery and/or blood draw is enabled.

### SUMMARY OF THE INVENTION

Provided herein is a vascular access device that includes a catheter insertion apparatus and a needleless access connector (NAC) unit. The catheter insertion apparatus includes a housing having a distal housing end and a proximal housing end and defining a longitudinal passage therein, a needle slideably mounted within the longitudinal passage and defining a needle lumen, and a catheter assembly having a frame and a catheter coupled to the frame, with the frame being slideably mounted within the housing to advance the catheter within the needle lumen. The catheter insertion apparatus is operable in an initial configuration where the needle extends distally from the distal housing end and the catheter is proximally retracted within the needle lumen, and is operable in an actuated configuration where the needle is retracted back into the longitudinal passage of the housing and the catheter extends distally from the distal housing end. The NAC unit is positioned at a proximal end of the frame of the catheter assembly, with the NAC unit providing an access port to the catheter.

In certain configurations, the NAC unit includes a connector portion having a proximal coupler, the proximal coupler comprising a split-septum NAC.

In certain configurations, the NAC unit further includes a side port provided on the connector portion, the side port in fluid communication with the proximal coupler via a lumen defined in the connector portion, and an extension tube coupled to the side port to provide a fluid path into the connector portion.

In certain configurations, the connector portion of the NAC unit comprises a flush enhancing feature.

In certain configurations, the NAC unit further includes a stabilization portion coupled to the connector portion, the stabilization portion configured to be placed in contact with a skin surface of a patient.

In certain configurations, positioning of the NAC unit at the proximal end of the frame of the catheter assembly provides a closed-system VAD.

In certain configurations, the frame has a distal frame end and a proximal frame end, with the frame including a push member positioned at the proximal frame end and a plurality of arms extending distally from the push member to the distal frame end, the plurality of arms engaging with the housing to form a sliding engagement between the frame and the housing. The push member is configured to be pushed distally to slide the plurality of arms into the housing and thereby advance the catheter within the needle lumen.

In certain configurations, the NAC unit is integrated with the push member of the frame.

In certain configurations, the NAC unit is separate from the catheter insertion apparatus and coupleable to the push member.

In certain configurations, the push member includes a luer connection formed thereon at the proximal frame end, and wherein the connector portion of the NAC unit comprises a distal coupler configured to mate with the luer connection, the distal coupler comprising a luer lock connection.

In certain configurations, the push member includes a side port formed therein adjacent the proximal end frame end.

In certain configurations, the NAC unit is connected to the side port of the push member via a length of intermediate tubing extending between the side port and a distal coupler of the NAC unit.

In certain configurations, the NAC unit is coupled to the push member with the catheter insertion apparatus in the initial configuration or in the actuated configuration.

In certain configurations, the access port and the catheter form an instrument compatible fluid path.

In certain configurations, in transitioning from the initial configuration to the actuated configuration, the push member is pushed distally and into contact with the housing, with the plurality of arms sliding into the housing as the push member is pushed distally.

Also provided herein is a catheter system including a vascular access device that includes a catheter insertion apparatus and a NAC unit. The catheter insertion apparatus includes a housing having a distal housing end and a proximal housing end and defining a longitudinal passage therein, a needle slideably mounted within the longitudinal passage and defining a needle lumen, and a catheter assembly having a frame and a catheter coupled to the frame, with the frame being slideably mounted within the housing to advance the catheter within the needle lumen. The catheter insertion apparatus is operable in an initial configuration where the needle extends distally from the distal housing end and the catheter is proximally retracted within the needle lumen, and is operable in an actuated configuration where the needle is retracted back into the longitudinal passage of the housing and the catheter extends through the needle lumen and distally from the distal housing end. The NAC unit is positioned at a proximal end of the frame of the catheter assembly, with the NAC unit providing an access port to the catheter. The catheter system also includes an instrument delivery device or blood draw device coupled to the access port of the NAC unit.

In certain configurations, the instrument delivery device is configured to advance an instrument through the NAC unit and into and out distally past the catheter.

In certain configurations, the instrument is one of a guidewire, sensor, wire, fiber, obturator, or tube.

In certain configurations, the instrument delivery device or blood draw device includes a lock configured to secure the instrument delivery device or blood draw device to the NAC unit.

### BRIEF DESCRIPTION OF THE DRAWINGS

FIG. 1 is a top view of a vascular access device, with a catheter insertion device thereof in an initial configuration, in accordance with an aspect of the disclosure;

FIG. 2 is a top view of the vascular access device of FIG. 1, with the catheter insertion device in an actuated configuration;

FIG. 3 is a top view of a vascular access device, with a catheter insertion device thereof in an initial configuration, in accordance with another aspect of the disclosure;

FIG. 4 is a top view of the vascular access device of FIG. 3, with the catheter insertion device in an actuated configuration;

FIG. 5 is a top view of a vascular access device, with a catheter insertion device thereof in an initial configuration, in accordance with another aspect of the disclosure;

FIG. 6 is a top view of the vascular access device of FIG. 5, with the catheter insertion device in an actuated configuration;

FIG. 7 is a top view of a vascular access device, with a catheter insertion device thereof in an initial configuration, in accordance with another aspect of the disclosure;

FIG. 8 is a top view of the vascular access device of FIG. 7, with the catheter insertion device in an actuated configuration;

FIG. 9 is a top view of a vascular access device, with a catheter insertion device thereof in an initial configuration, in accordance with another aspect of the disclosure;

FIG. 10 is a top view of the vascular access device of FIG. 9, with the catheter insertion device in an actuated configuration; and

FIG. 11 is a top view of a vascular access device coupled to an instrument delivery device, in accordance with another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

Provided herein are embodiments of vascular access device for inserting a catheter into a patient's vascular system, such as a peripheral intravenous catheter (PIVC), with the vascular access device further configured to provide for introducing instruments through the catheter via a near-patient access port thereon.

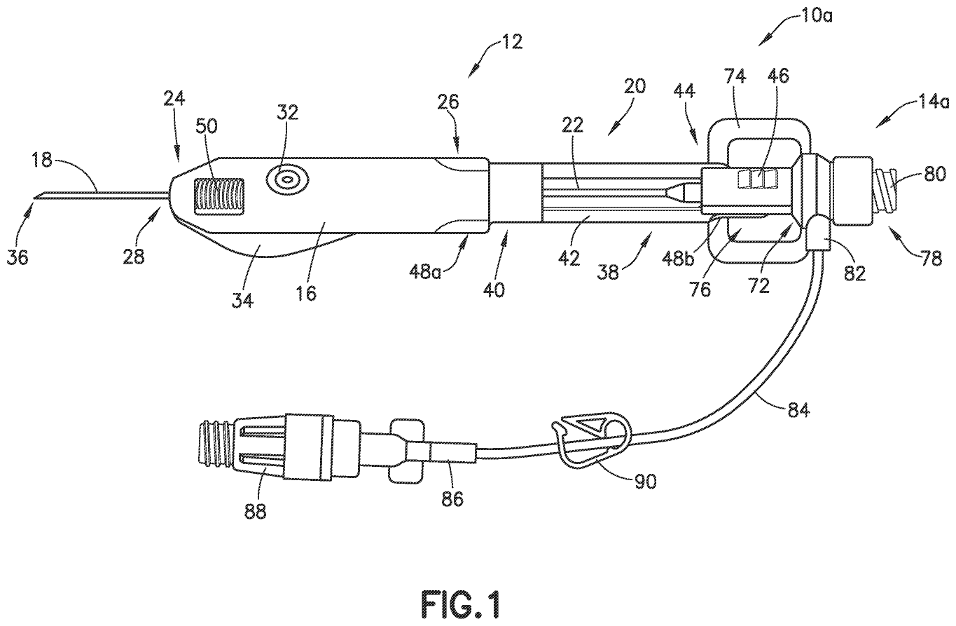

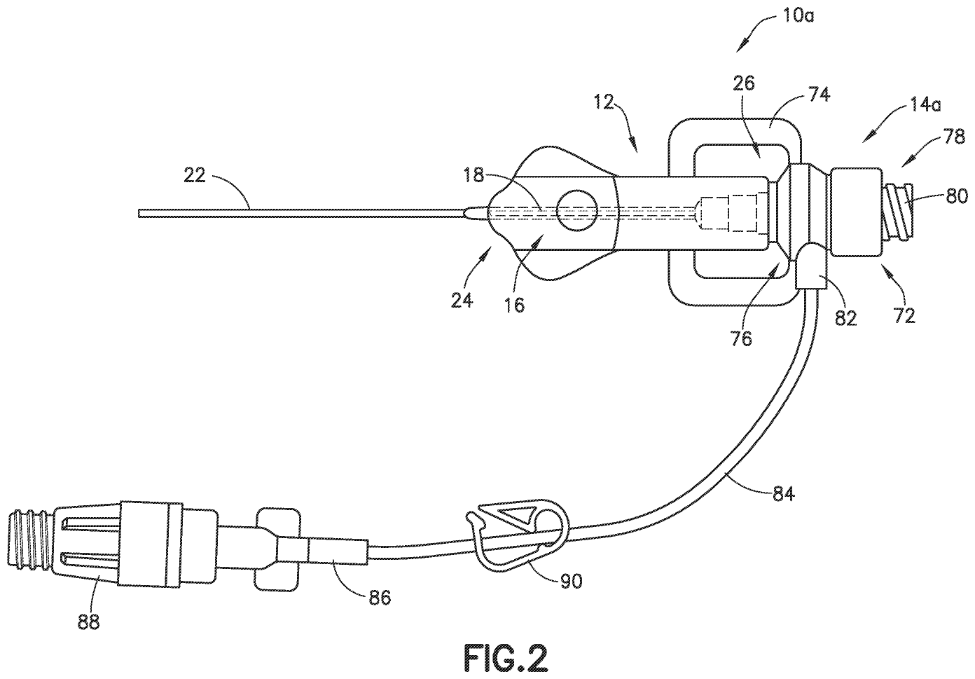

Referring first to FIGS. 1 and 2, a non-limiting embodiment of a vascular access device (VAD) 10a is shown in accordance with one aspect of the disclosure. The VAD 10a includes a catheter insertion apparatus 12 and a needleless access connector (NAC) unit 14a, with the VAD 10a configured to provide for integration or connection of the NAC unit 14a with the catheter insertion apparatus 12. As will be explained in further detail below, the NAC unit 14a provides a near-patient access port for the VAD 10a through which instrument delivery and/or blood draw is enabled, with such instrument delivery and/or blood draw achieved with the catheter insertion apparatus 12 remaining intact at the insertion site of the catheter.

With reference first to the catheter insertion apparatus 12 of VAD 10a, the catheter insertion apparatus 12 generally includes a housing 16, a needle 18, and a catheter assembly 20, with the catheter assembly 20 including a venous access catheter 22 therein. The catheter insertion apparatus 12 is operable in each of an initial configuration (FIG. 1) and an actuated configuration (FIG. 2). In the initial configuration of the catheter insertion apparatus 12 (i.e., the configuration used for initial needle penetration into the patient's vein), the needle 18 is positioned to extend out distally from housing 16, while the catheter 22 is fully retracted within the needle 18 and the housing 16. In the actuated configuration of the catheter insertion apparatus 12 (i.e., the configuration used for insertion of the catheter into the patient's vein and withdrawal/retraction of the needle), the catheter 22 is advanced through the needle 18 to extend distally out from the needle 18 and from the housing 16, while the needle 18 is fully retracted within the housing 16. The catheter insertion apparatus 12 is thus configured as an “over-the-catheter” type insertion apparatus, where the introducer needle 18 is positioned over the catheter 22 when advancing the catheter 22 into the vasculature of a patient.

The housing 16 of catheter insertion apparatus 12 is configured as an elongate member having a distal housing end 24 and a proximal housing end 26, with the distal housing end including a distal port 28 and the proximal housing end being open. The housing 16 defines a longitudinal passage 30 therein within which the needle 18 and portions of the catheter assembly 20 may be received. In some embodiments, a flashback window 32 and a pair of taping wings 34 may be provided on the housing 16. The flashback window 32 allows a user to detect when blood flows back into the device after a sharpened distal tip 36 of needle 18 first enters a vein, confirming that access has been achieved. The taping wings 34 facilitate taping, wrapping, or otherwise securing the housing 16 to the patient after the catheter 22 has been positioned in the target vein.

The catheter assembly 20 of catheter insertion apparatus 12 includes the venous access catheter 22 and a frame 38 that retains the catheter, with the frame 38 having a distal end 40 that includes a plurality of arms 42 receivable within the housing 16 and a proximal end 44 that includes a push member 46 and with which the NAC unit 14a is integrated, as explained in greater detail below. In the initial configuration of the catheter insertion apparatus 12, the push member 46 of frame 38 is spaced apart from the proximal housing end 26, while being slideably attached to the housing 16 by the arms 42 of frame 38. The arms 42 engage the housing 16 in a sliding manner, for advancing the catheter assembly 20 into the interior passage 30 of the housing 16 as the push member 46 is pushed distally by a user.

In operation of the of catheter insertion apparatus 12, the needle 18 is initially introduced into a vein with the catheter insertion apparatus 12 in its initial configuration, with introduction of the needle 18 performed, for example, by a manual placement in a conventional manner. After introduction of the needle 18 into the vein, flashback will be observed through the window 32, and the catheter 22 can be distally advanced from the needle 18 (and distally out past housing 16) by distally advancing the push member 46, as shown in FIG. 2. That is, the push member 46 may be actuated in the distal direction until it comes into contact with the proximal end 26 of housing 16.

According to some embodiments, upon the catheter 22 being advanced (but with the needle 18 still being in an initial extending distally from the housing 16), the push member 46 may be rotated or further actuated relative to the housing 16 to retract the needle 18 and lock the catheter 22 in place. In one embodiment, gripping features 48a and 48b may be provided on the housing 16 and the push member 46, respectively, with the needle 18 being retracted and the catheter 22 locked upon the gripping features 48a and 48b coming into axial alignment with each other. Upon rotation of the push member 46 relative to the housing 16, the catheter insertion apparatus 12 is thus placed in the actuated configuration, with the catheter 22 positioned distally from the needle 18 and the housing 16, and with the needle 18 retracted back into the housing 16.

It is recognized that the catheter insertion apparatus 12 could include various internal components that provide for the retraction of the needle 18 and the locking of the catheter 22 in place when the catheter insertion apparatus 12 is in the actuated configuration. As an example, the catheter insertion apparatus 12 may include a coil spring 50 and a plurality of locking features (not shown) provided on the needle 18 (i.e., on a hub thereof) and also within housing 16 and on frame 38, that interact with each other to provide for such retraction and locking functions. The coil spring 50 may be concentrically positioned about the needle 18 and between the distal housing end 24 and a hub of the needle (provided on a proximal end thereof), with the coil spring 50 initially retained in a compressed state and then allowed to expand to a relaxed state upon the catheter insertion apparatus 12 transitioning from the initial configuration to the actuated configuration, so as to cause the needle 18 to retract back into the housing 16. A first group of locking features (on needle 18 and housing 16) may be utilized to allow for the coil spring 50 to be released from its compressed state and expand to its relaxed state, thereby retracting needle 18 back into housing 16, while a second group of locking features (on needle 18 and frame 38) may be utilized to then lock the catheter 22 in place after retraction of the needle 18.

As indicated above, the VAD 10a includes a NAC unit 14a that is integrated with or connected to the catheter insertion apparatus 12. In the illustrated embodiment, the NAC unit 14a is integrated with the catheter insertion apparatus 12, such that the catheter insertion apparatus 12 and NAC unit 14a are provided as a single device. In the illustrated non-limiting embodiment, the NAC unit 14a has a connector portion 72 and a stabilization portion 74, with the connector portion 72 integrated with the catheter insertion apparatus 12 and the stabilization portion 74 coupled to the connector portion 72 and configured to be placed in contact with the skin surface of a patient at or near an insertion site of catheter 22.

The connector portion 72 is configured such that a distal end 76 thereof is integrated with the catheter insertion apparatus 12, such as being formed integrally with push member 46 and/or irremovably coupled thereto, and such that a lumen (not shown) defined by the connector portion 72 is at least selectively in fluid communication with the catheter 22 of catheter insertion apparatus 12. A proximal end 78 of the connector portion 72 has a coupler 80 that provides a proximal port on the NAC unit 14a. The proximal coupler 80 may be configured as a split-septum NAC configured to receive a blunt introducer of a peripheral device such as, e.g., a blood draw device (e.g., PIVO™ from Becton, Dickinson and Company), or a vascular access probe (VAP) for in-vein digital measurement of patient data such as temperature, pH, lactate, and/or other blood-based measurements, so as to be physically and fluidically coupled thereto.

The connector portion 72 also includes and/or defines one or more additional ports, such as a side port 82. Side port 82 can be included on the connector portion 72 so as to be located between the proximal end 78 and the distal end 76. In some embodiments, the arrangement of side port 82 can be such that the connector portion 72 forms, for example, a Y-connector or a T-connector. More particularly, side port 82 can be disposed substantially perpendicular (e.g., about 90 degrees) to the lumen of the connector portion 72 and near or adjacent the proximal coupler 80. In non-limiting embodiments, side port 82 extends from connector portion 72 at an angle, relative to the lumen that is not 90 degrees (e.g., side port 82 extends at an angle of, for example and without limitation, 15-165 degrees, with all values and subranges therebetween inclusive).

In the embodiment of FIGS. 1 and 2, an extension tubing 84 is integrated with the NAC unit 14a and is joined thereto via the side port 82. The extension tubing 84 is in fluid communication with a lumen of the side port 82. In some embodiments, the side port 82 and/or the extension tubing 84 can be and/or can form at least a portion of a fluid line that can be used to deliver fluid, remove fluid, flush fluid, and/or the like. In such embodiments, for example, an arrangement in which side port 82 is disposed adjacent to the proximal coupler 80 can enable flushing of the proximal coupler 80, any valve(s) included therein, and/or a space between any valve(s) and an inner surface of the connector portion 72 (e.g., defining at least a portion of the lumen).

As shown further shown in FIGS. 1 and 2, a proximal access port 86 is coupled to a proximal end portion of the extension tubing 84, with the extension tubing 84 in fluid communication with the side port 82 of the catheter connector 46. A connector 88 of a desired type (e.g., NAC) may be coupled to the access port 86 to allow for fluid connection of a component (e.g., syringe, IV bag, etc.) to the extension tubing 84. In some embodiments, a clamp 90 is provided on the extension tubing 84 to selectively restrict flow through the extension tubing 84.

Beneficially, integration of the NAC unit 14a with the catheter insertion apparatus 12 provides a closed catheter system with a near-patient access port for the VAD 10a through which instrument delivery and/or blood draw is enabled. The near-patient access port provided by the NAC unit 14a allows for guidewire, sensor, wires, fiber, obturators, or other instruments to be advanced in a straight line into the fluid path of the VAD 10a or beyond the tip of the catheter.

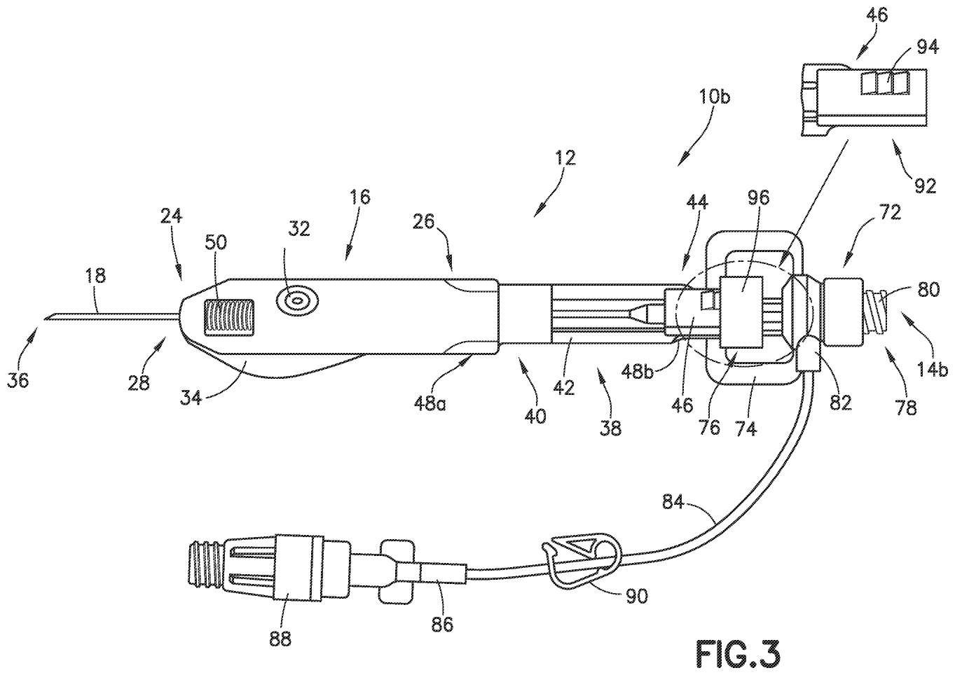

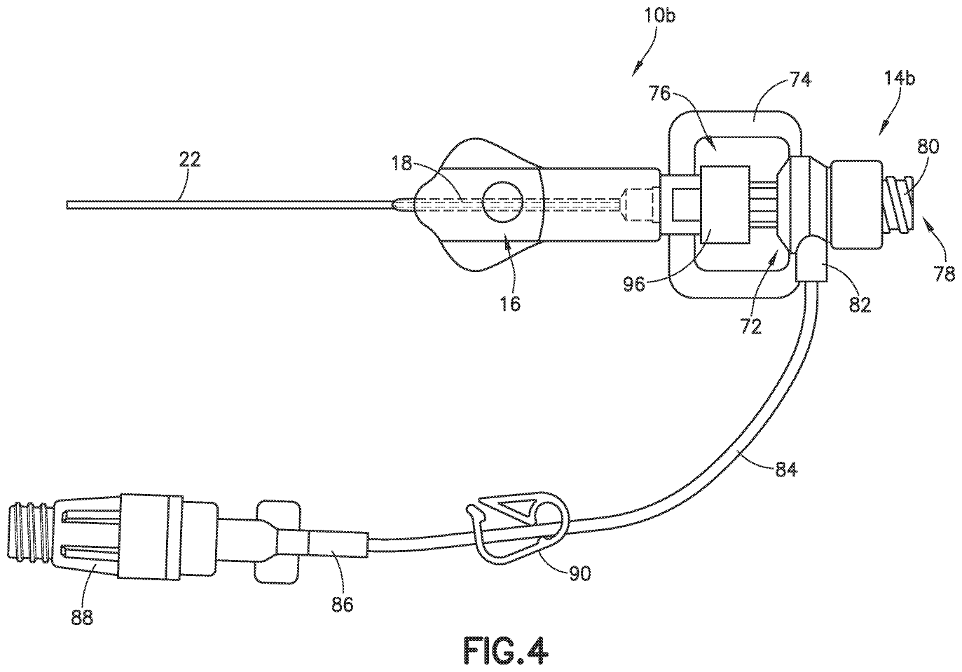

Referring now to FIGS. 3 and 4, a VAD 10b is shown in accordance with another aspect of the disclosure. The VAD 10b of FIGS. 3 and 4 differs from the VAD 10a of FIGS. 1 and 2 in that an NAC unit 14b is provided as a removable unit that is separate from the catheter insertion apparatus 12 but coupleable thereto. In such an embodiment, the push member 46 of catheter insertion apparatus 12 includes a connector 92 at the proximal end thereof that provides for coupling of the NAC unit 14b thereto. The proximal connector 92 of the push member 46 may thus, in one embodiment, be configured as a female luer connection having threads 94 on an outer surface thereof.

In the embodiment of FIGS. 3 and 4, the NAC unit 14b is structured as previously described in the embodiment of FIGS. 1 and 2, in that the NAC unit 14b includes a connector portion 72 and a stabilization portion 74, with the connector portion 72 including a proximal coupler 80 and a side port 82. As previously described, the proximal coupler 80 is configured as a NAC that provides a proximal port on the NAC unit 14b and that may be configured as a split-septum NAC configured to receive a blunt introducer of a peripheral device, while the side port 82 provides for an extension tube 84 (or extension set) to be coupled thereto or integrally formed therewith. The NAC unit 14b additionally includes a distal coupler 96 thereon (positioned at distal end 76) configured to mate with the connector 92 of push member 46. According to one embodiment, the distal coupler 96 is configured as a male luer connection that mates with the female luer connection of connector 92. Coupling of the NAC unit 14b to the catheter insertion apparatus 12 (via mating of distal coupler 96 with connector 92) thus provides a closed catheter system with a near-patient access port for the VAD 10b through which instrument delivery and/or blood draw is enabled. The near-patient access port provided by the NAC unit 14b allows for guidewire, sensor, wires, fiber, obturators, or other instruments to be advanced in a straight line into the fluid path of the VAD 10b or beyond the tip of the catheter 22.

According to embodiments, the NAC unit 14b may be attached to the catheter insertion apparatus 12 with the catheter insertion apparatus 12 in the initial configuration or the actuated configuration, i.e., prior to or after catheter insertion and advancement. The selective connection of the NAC unit 14b in this matter provides flexibility to a clinician regarding the timing of connection of the NAC unit 14b to the catheter insertion apparatus 12, so as to provide for ease of use of the catheter insertion apparatus 12 while also enabling easy introduction/advancement of an instrument into and through the VAD 10b.

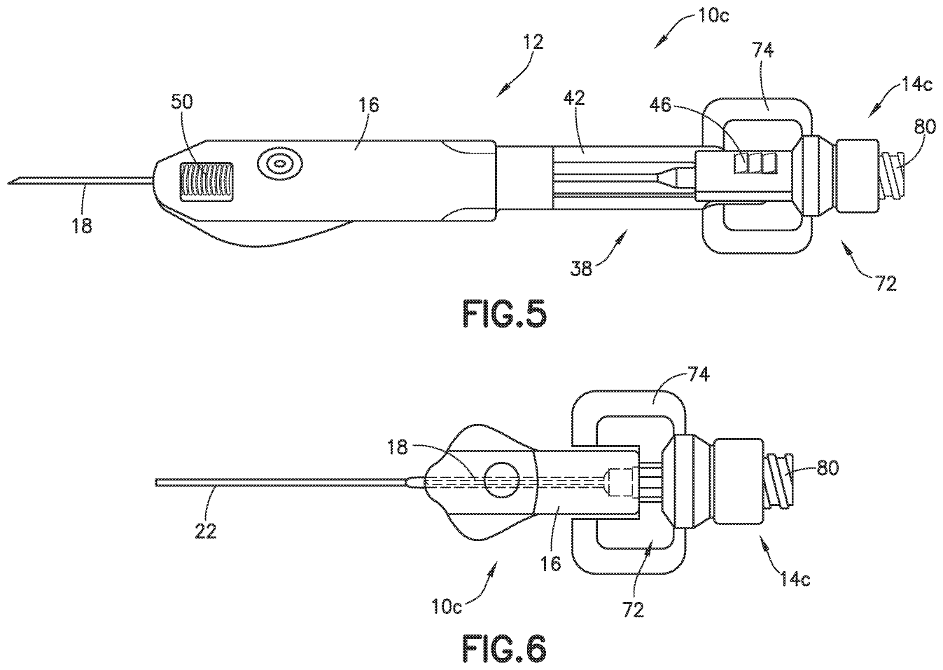

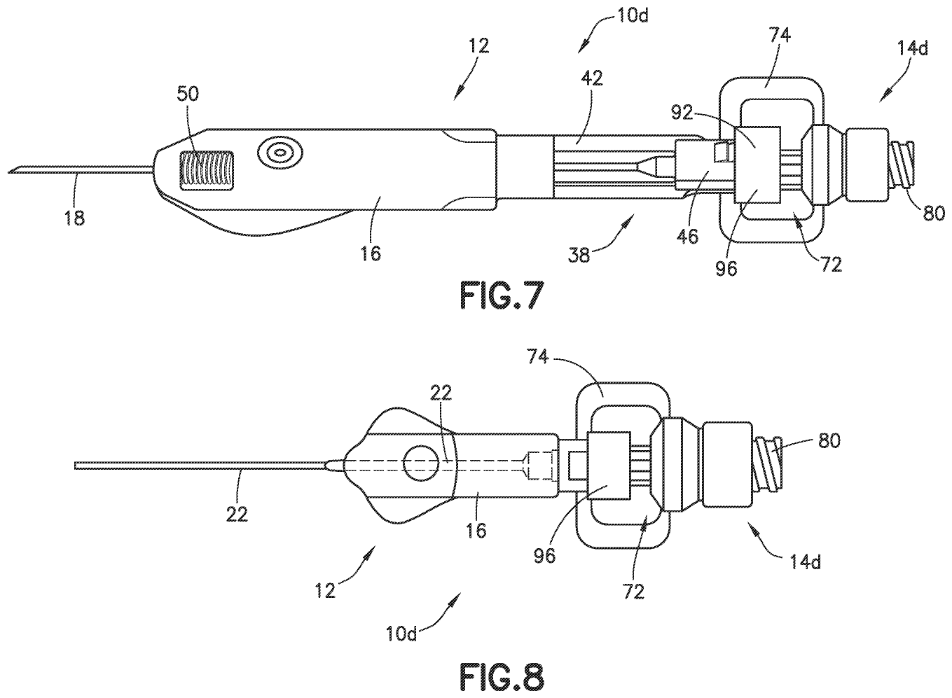

Referring now to FIGS. 5 and 6 and FIGS. 7 and 8, VADs 10c, 10d are shown in accordance with additional aspects of the disclosure. The VAD 10c of FIGS. 5 and 6 is identical to the VAD 10a of FIGS. 1 and 2, except that an NAC unit 14c integrated with the catheter insertion apparatus 12 does not include a side port and associated extension tube/set integrated therewith, as does the NAC unit 14a of VAD 10a. Similarly, the VAD 10d of FIGS. 7 and 8 is identical to the VAD 10b of FIGS. 3 and 4, except that a separate/attachable NAC unit 14d thereof (that is attachable to the catheter insertion apparatus 12) does not include a side port and associated extension tube/set integrated therewith, as does the NAC unit 14b of VAD 10b. In either of the embodiments, the integration or connection of the NAC unit 14c, 14d to the catheter insertion apparatus 12 provides a closed system VAD 10c, 10d with a near-patient access port for instrument delivery and blood draw.

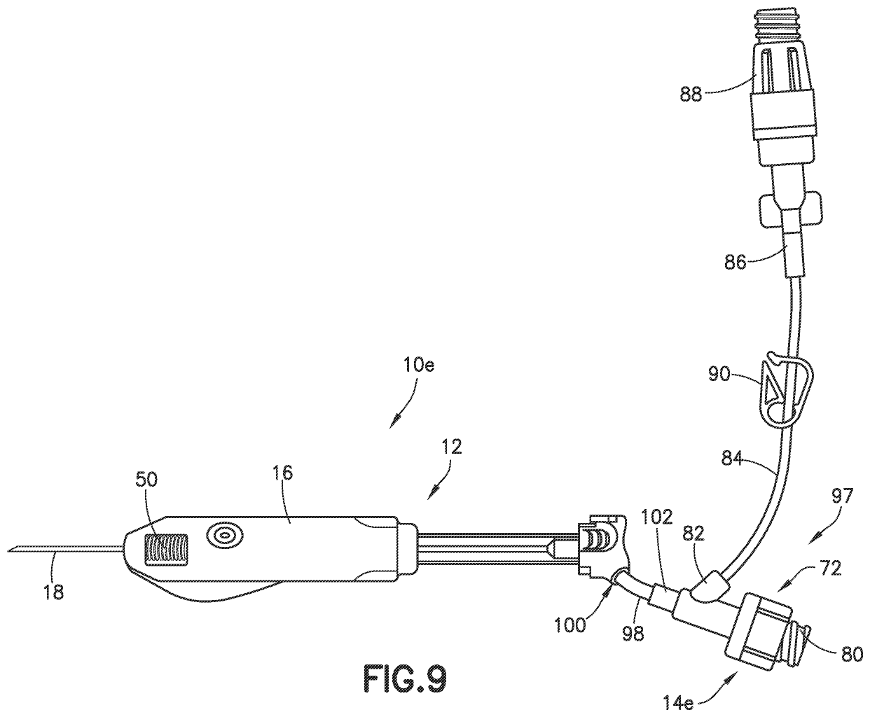

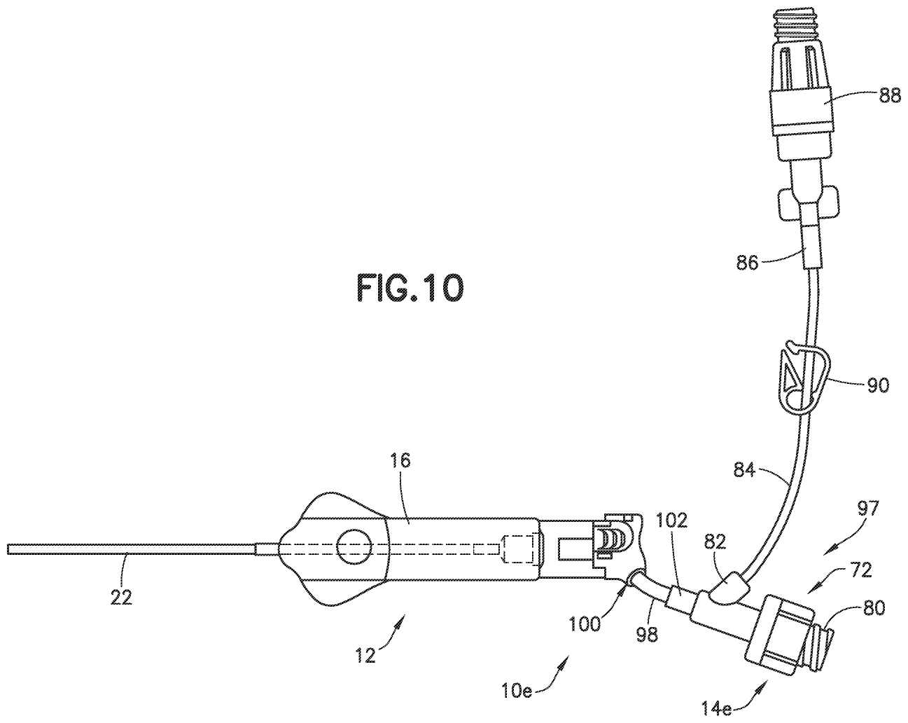

Referring now to FIGS. 9 and 10, a VAD 10e is shown in accordance with another aspect of the disclosure. The VAD 10e of FIGS. 9 and 10 differs from the VADs 10a, 10b, 10c, 10d of FIGS. 1-8 in that an NAC unit 14e thereof is provided as part of an extension set 97 that is separate from the catheter insertion apparatus 12 and coupled thereto via an intermediate tubing 98. To accommodate connection of the extension set 97 (including NAC unit 14e) to the catheter insertion apparatus 12, the push member 46 of catheter insertion apparatus 12 includes a side port 100 adjacent the proximal end thereof in which intermediate tubing 98 is received, with the intermediate tubing 98 extending between the side port 100 and the extension set 97 and NAC unit 14e.

In the embodiment of FIGS. 9 and 10, the NAC unit 14e is structured similarly to the NAC units 14a, 14b previously described in the embodiments of FIGS. 1-4, in that the NAC unit 14e includes a connector portion 72 including a proximal coupler 80 configured as an NAC and a side port 82 to which extension tubing is coupled (or integrally formed with). However, NAC unit 14e includes a distal coupler 102 configured to receive the intermediate tubing 98 therein, so as to fluidly connect an interior lumen (not shown) of connector portion 72 with the side port 100 on catheter insertion apparatus 12 and, in turn, the catheter 22 of catheter insertion apparatus 12. In some embodiments, a stabilization portion (not shown) may be provide on NAC unit 14e.

Coupling of the NAC unit 14e (as part of extension set 97) to the catheter insertion apparatus 12, via side port 100 on push member 46 and intermediate tubing 98, thus provides a closed catheter system with a near-patient access port for the VAD 10e through which instrument delivery and/or blood draw is enabled. The near-patient access port provided by the NAC unit 14e allows for guidewire, sensor, wires, fiber, obturators, or other instruments to be advanced into the fluid path of the VAD 10e or beyond the tip of the catheter 22.

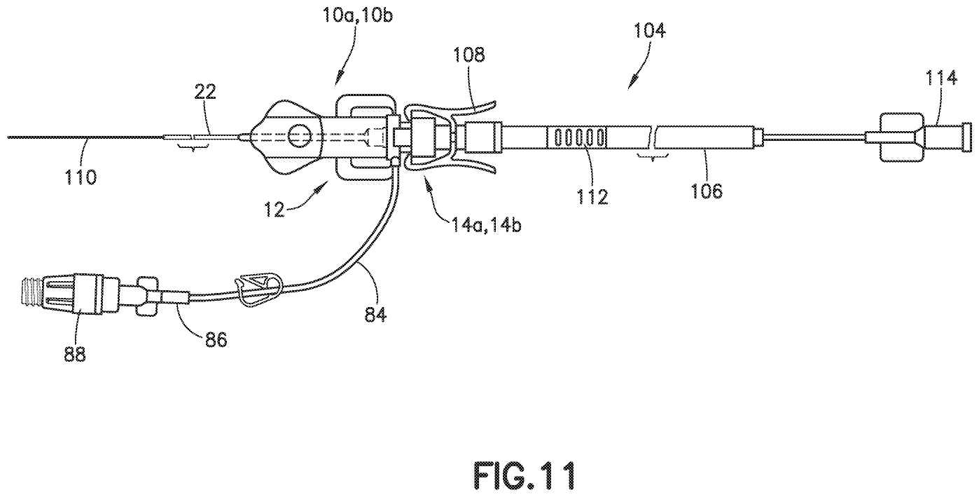

Referring now to FIG. 11, the VAD 10a, 10b of FIGS. 1-2 or FIGS. 3-4 is shown with an instrument advancement device 104 coupled thereto, so as to illustrate connection of a device to the near-patient access port thereof enabled by inclusion of the NAC unit 14a, 14b in the VAD 10a, 10b. The instrument advancement device 104 can be any suitable shape, size, or configuration and can be coupled to the VAD 10a, 10b via the NAC unit 14a, 14b. As shown in FIG. 11, the instrument advancement device 104 includes an introducer 106, a lock 108, an instrument 110 (e.g., catheter, guidewire, probe, etc.), and an actuator 112. A portion of the actuator 112 is configured to be advanced along a top surface of the introducer 106, which in turn, facilitates the advancing of the instrument 110 through the introducer 106, the lock 108, the VAD 10a, 10b, and distally past the catheter 22. In some embodiments, a luer adapter, luer lock access device (LLAD), or other connecting device 114 may be provided at a proximal end of the introducer 106 to enable connection of another component to the device, such as a syringe or vacutainer to facilitate a blood draw.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A vascular access device, comprising:
a catheter insertion apparatus including:
a housing having a distal housing end and a proximal housing end and defining a longitudinal passage therein;
a needle slideably mounted within the longitudinal passage and defining a needle lumen; and
a catheter assembly comprising a frame and a catheter retained by the frame, the frame being slideably mounted within the housing to advance the catheter within the needle lumen;
wherein the catheter insertion apparatus is operable in an initial configuration where the needle extends distally from the distal housing end and the catheter is proximally retracted within the needle lumen, and is operable in an actuated configuration where the needle is retracted back into the longitudinal passage of the housing and the catheter extends through the needle lumen and distally from the distal housing end; and
a needleless access connector (NAC) unit positioned at a proximal end of the frame of the catheter assembly, the NAC unit providing an access port to the catheter.

2. The vascular access device of claim 1, wherein the NAC unit comprises a connector portion including a proximal coupler, the proximal coupler comprising a split-septum NAC.

3. The vascular access device of claim 2, wherein the NAC unit further comprises:
a side port provided on the connector portion, the side port in fluid communication with the proximal coupler via a lumen defined in the connector portion; and
an extension tube coupled to the side port to provide a fluid path into the connector portion.

4. The vascular access device of claim 3, wherein the connector portion of the NAC unit comprises a flush enhancing feature.

5. The vascular access device of claim 2, wherein the NAC unit further comprises a stabilization portion coupled to the connector portion, the stabilization portion configured to be placed in contact with a skin surface of a patient.

6. The vascular access device of claim 1, wherein positioning of the NAC unit at the proximal end of the frame of the catheter assembly provides a closed-system VAD.

7. The vascular access device of claim 2, wherein the frame has a distal frame end and a proximal frame end, the frame comprising:
a push member positioned at the proximal frame end; and
a plurality of arms extending distally from the push member to the distal frame end, the plurality of arms engaging with the housing to form a sliding engagement between the frame and the housing;
wherein the push member is configured to be pushed distally to slide the plurality of arms into the housing and thereby advance the catheter within the needle lumen.

8. The vascular access device of claim 7, wherein the NAC unit is integrated with the push member of the frame. 9 The vascular access device of claim 7, wherein the NAC unit is separate from the catheter insertion apparatus and coupleable to the push member.

10. The vascular access device of claim 9, wherein the push member includes a luer connection formed thereon at the proximal frame end, and wherein the connector portion of the NAC unit comprises a distal coupler configured to mate with the luer connection, the distal coupler comprising a luer lock connection.

11. The vascular access device of claim 9, wherein the push member includes a side port formed therein adjacent the proximal end frame end.

12. The vascular access device of claim 11, wherein the NAC unit is connected to the side port of the push member via a length of intermediate tubing extending between the side port and a distal coupler of the NAC unit.

13. The vascular access device of claim 9, wherein the NAC unit is coupled to the push member with the catheter insertion apparatus in the initial configuration or in the actuated configuration.

14. The vascular access device of claim 1, wherein the access port and the catheter form an instrument compatible fluid path.

15. The vascular access device of claim 1, wherein in transitioning from the initial configuration to the actuated configuration, the push member is pushed distally and into contact with the housing, with the plurality of arms sliding into the housing as the push member is pushed distally.

16. A catheter system, comprising:
a catheter insertion apparatus including:
a housing having a distal housing end and a proximal housing end and defining a longitudinal passage therein,
a needle slideably mounted within the longitudinal passage and defining a needle lumen, and
a catheter assembly comprising a frame and a catheter retained by the frame, the frame being slideably mounted within the housing to advance the catheter within the needle lumen,
wherein the catheter insertion apparatus is operable in an initial configuration where the needle extends distally from the distal housing end and the catheter is proximally retracted within the needle lumen, and is operable in an actuated configuration where the needle is retracted back into the longitudinal passage of the housing and the catheter extends through the needle lumen and distally from the distal housing end, and
a needleless access connector (NAC) unit positioned at a proximal end of the frame of the catheter assembly, the NAC unit providing an access port to the catheter; and
an instrument delivery device or blood draw device coupled to the access port of the NAC unit.

17. The catheter system of claim 16, wherein the instrument delivery device or blood draw device is configured to advance an instrument through the NAC unit and into and out distally past the catheter.

18. The catheter system of claim 17, wherein the instrument comprises one of a guidewire, sensor, wire, fiber, obturator, or tube.

19. The catheter system of claim 16, wherein the instrument delivery device or blood draw device comprises a lock configured to secure the instrument delivery device or blood draw device to the NAC unit.
